(19) United States
(12) Patent Application Publication
(10) Pub. No.: US 2016/0374975 A1
(43) Pub. Date: Dec. 29, 2016

(54) COMPOSITION FOR TREATMENT OF NAFLD

(71) Applicant: Pronova BioPharma Norge AS, Lysaker (NO)

(72) Inventor: Carola Rosseland, Lysaker (NO)

(73) Assignee: Pronova BioPharma Norge AS, Norway (NO)

(21) Appl. No.: 15/189,177

(22) Filed: Jun. 22, 2016

(30) Foreign Application Priority Data
Jun. 26, 2015 (NO) 20150838

(51) Int. Cl. A61K 31/202

(52) U.S. Cl. CPC A61K 31/202

(57) ABSTRACT

A composition and method of treating patients diagnosed with NAFLD is disclosed. The composition contains n-3 polyunsaturated fatty acids (PUFAs) for treatment of NAFLD patients, wherein the amount of PCB 153 in the composition has been minimized. The composition is administered to a patient in a sufficient amount and for a sufficient time to increase the level of n-3 PUFAs or to correct a deficiency of n-3 PUFAs in the patient's blood. The method increases the level of n-3 PUFAs without contributing to the body burden of PCB 153.

### CROSS-REFERENCE TO RELATED APPLICATIONS

This application claims the benefit of Norwegian patent application No. 20150838, filed Jun. 26, 2015.

### FIELD OF THE INVENTION

The present invention relates to a composition for treatment of patients diagnosed with non-alcoholic fatty liver disease (NAFLD). Particularly, the invention provides a composition of n-3 polyunsaturated fatty acids (n-3 PUFAs) wherein the amount of PCB 153 has been minimized, as it has been found that this specific PCB congener is a driver for the pathophysiology of NAFLD. The invention further provides a composition for use in therapy of patients diagnosed with NAFLD. Yet further, the invention provides a method to increase the level of n-3 PUFAs or to correct a deficiency of n-3 PUFAs in NAFLD patients' blood without increasing the body burden of PCB 153.

### BACKGROUND OF THE INVENTION

NAFLD is one form of fatty liver, occurring when fat is deposited (steatosis) in the liver due to causes other than excessive alcohol use. NAFLD is the most common liver disorder in Western industrialized nations. It is a common cause of chronic liver disease (CLD) in North America and a growing contributor to the burden of CLD requiring liver transplantation. It has been shown to increase risk of hepatocellular carcinoma, type 2 diabetes, and cardiovascular diseases. NAFLD encompasses both non-alcoholic fatty liver (NAFL) and non-alcoholic steatohepatitis (NASH), with the latter being the more serious form of the disease. The relatively long asymptomatic time interval in the progression of NAFL to NASH to cirrhosis and ultimately liver failure represents significant challenges in the development of treatments, and presently no FDA approved therapies for NASH exist. In September, 2013, the U.S FDA and the American Association for the Study of Liver Diseases (AASLD) jointly sponsored a workshop to tackle this challenge. The joint workshop pointed out the substantial knowledge gap that exists regarding disease modifiers, and the urgent need to develop methods that identify, and improve the health of, populations at particular risk.

The terms non-alcoholic fatty liver disease (NAFLD) and non-alcoholic steatohepatitis (NASH) frequently are used interchangeably despite the fact that NAFLD encompasses a much broader spectrum of liver disease including simple hepatosteatosis (>5% of hepatocytes histologically). Hepatosteatosis, also called NAFL, is most likely a relatively benign disorder when not accompanied by an inflammatory response and cellular damage. However, a subgroup of NAFLD patients have liver cell injury and inflammation in addition to hepatosteatosis, a condition known as non-alcoholic steatohepatitis (NASH).

NAFL is related to insulin resistance and metabolic syndrome (MetS) and is defined by the presence of more than 5% hepatic steatosis. Lifestyle changes including weight loss may ameliorate liver fat, but there is a need for additional treatment methods. Although treatments originally developed for other insulin-resistant states, including metformin and thiazolidinediones, may have some effect, there are concerns about long-term use of these agents. Accordingly, there is a need for compositions and methods for treating NAFLD.

Omega-3 fatty acid treatment is safe and has attracted interest as a potential treatment of NAFLD. For instance, Scorletti et al, “Effects of purified Eicosapentaenoic and Docosahexaenoic Acids in Non-alcoholic Fatty Liver Disease: Results from the WELCOME study”, Hepatology, 2014, shows that erythrocyte DHA enrichment with DHA and EPA treatment is linearly associated with decreased liver fat percentage. Also, Lou et al, “Serum phospholipid omega-3 polyunsaturated fatty acids and insulin resistance in type 2 diabetes mellitus and non-alcoholic fatty liver disease”, Journal of Diabetes and Its Complications, 28 (2014), 711-714, have shown that in a clinical study designed to assess the relationship between serum phospholipid omega-3 polyunsaturated fatty acids (PUFA) and NAFLD patients, the total n-3 PUFA levels were significantly lower in the NAFLD group compared to the control group (6.97±2.32% vs 10.08±2.76%, p<0.05). Oxidative stress and hepatic mitochondria play a role in the pathogenesis of NAFLD and liver glutathione (GSH) is diminished, as shown by Oliveira et al, “Liver mitochondrial dysfunction and oxidative stress in the pathogenesis of experimental nonalcoholic fatty liver disease”, Braz J Med Biol Res 39(2), 2006. Omega-3 PUFA supplementation reduces lipoperoxidation in steatic liver, reduces hepatic liver content, and increases GSH. Further, patent applications suggest combinations of different omega-3 fatty acids may have a positive effect in the treatment of NAFLD. WO2014/142364 discloses a method for treating fatty liver disease or disorder including administration of a therapeutically effective amount of a composition comprising eicosapentaenoic acid (EPA) or ethyl-EPA.

Polychlorinated biphenyls (PCBs) are persistent environmental pollutants detectable in the serum of all American adults. Cave et al, “Polychlorinated Biphenyls, Lead, and Mercury are associated with liver disease in American adults; NHanes 2003-2004”, Environmental Health Perspectives, Volume 18, No 12, 2010, report that exposure to PCB, lead, and mercury is associated with unexplained alanine transaminase (ALT) elevation, which have been associated with non-alcoholic fatty liver disease (NAFLD) in epidemiologic studies. Among the possible 209 PCB congeners, polychlorinated biphenyl 153 (PCB 153) has the highest serum level. Wahlang et al, “Polychlorinated biphenyl 153 is a diet-dependent obesogen that worsens nonalcoholic fatty liver disease in male C57BL6/J mice”, Journal of Nutritional Biochemistry 24 (2013), 1587-1595 reports that PCB 153 exposure in high-fat-fed mice was associated with increased visceral adiposity, hepatic steatosis, and plasma adipokines. Likewise, expression of hepatic genes implicated in β-oxidation was reduced while the expression of genes associated with lipid biosynthesis was increased. Similar results were not seen in mice exposed to high fat diet (HFD) alone or PCB 153 alone, demonstrating a synergistic interaction between this particular PCB congener and HFD resulting in dramatically increased obesity and NAFLD. Thus, PCB 153 is an obesogen that exacerbates hepatic steatosis, alters adipocytokines, and disrupts normal hepatic lipid metabolism when administered with a high fat diet. Because there is a synergistic interaction between PCB 153 and a high fat diet (HFD), obesity and NAFL may be increased in individuals exposed to PCB 153 compared to those without exposure. The mechanism for this effect is partly revealed by Shi et al, “Metabolic analysis of the effects of polychlorinated biphenyls in nonalcoholic fatty liver disease”, J. Proteome Res. 2012, 3805-3815, which shows that PCB 153 combined with HFD significantly changes hepatic metabolism. Mice exposed to HFD alone and HFD combined with PCB 153 show substantial differences in hepatic levels of 14 metabolites. For example, glutathione in HFD+PCB 153 treated mice was reduced 6-fold compared with HFD treatment alone. Hence, PCB 153 is a diet toxin that affects metabolic pathways.

Because all US adults are exposed to PCB 153, this particular nutrient-toxicant interaction potentially impacts human obesity/NAFLD.

Compositions comprising high amounts of omega-3 are concentrates commonly derived from crude fish oil, which naturally contain different types of fatty acids. The starting material, i.e. crude fish oil, also contains various persistent organic pollutants (POPs), such as dioxins, PCBs, and brominated flame retardants (BFRs). Because of the physicochemical properties of the lipophilic POPs, they accumulate in the marine environment and human exposure mainly derives from dietary intake of fish and fish oils. Furthermore, processes that concentrate the polyunsaturated fatty acids will also concentrate the lipophilic pollutants if they are not removed in the manufacturing process.

Numerous processes disclosed in the prior art address how to obtain concentrates of omega-3 fatty acids from crude oils, and several patents and patent applications disclose methods to purify fish oils and reduce the concentrations of POPs. WO2004/007654 describes a process for decreasing environmental pollutants in an oil or fat using a working fluid. Further, EP 2 438 819 provides a process for obtaining concentrates of eicosapentaenoic acid (EPA) and docosahexaenoic acid (DHA) esters from marine oils comprising a content of POPs below the mandatory upper limits. Further, U.S. Pat. No. 8,258,330 discloses carrier fluid compositions and their use in processes for reducing the concentration of POPs in fish oils, reporting concentrations of different dioxins, furans, cholesterol, PCBs, polybrominated diphenyl ethers (PBDE), chlorinated hydrocarbons, and polycyclic aromatic hydrocarbons (PAHs) using the process. None of these disclosures however provide a teaching about specific compositions of omega-3 fatty acids, such as after processing, separation, and up-concentration, or the reduction of specific POPs, for use of a composition for a given indication such as NAFLD.

### BRIEF SUMMARY OF THE INVENTION

For the reasons stated above, applicant has identified that for the vulnerable group of NAFLD patients, being particularly sensitive to a certain PCB, there is a need for a highly purified omega-3 composition for use in a treatment of this patient group. Particularly, as the body may have higher vulnerability towards a specific PCB, i.e., PCB 153, than other PCBs, and as this particular PCB has a negative effect on NAFLD, applicant has found that omega-3 polyunsaturated fatty acid (PUFA) compositions for treatment of subjects with NAFLD should comprise a minimal amount of this PCB.

Based on the need for compositions and methods for treating patients diagnosed with NAFLD, and the new information that this patient group is particularly vulnerable to one specific PCB, the invention provides new n-3 PUFA compositions with very low content of PCB 153, and methods using these.

Accordingly, in a first aspect, the invention provides a composition comprising at least 40% of at least one of (all-Z omega-3)-5,8,11,14,17-eicosapentaenoic acid (EPA) and (all-Z omega-3)-4,7,10,13,16,19-docosahexaenoic acid (DHA), or derivatives thereof, by weight of the fatty acids therein, and wherein the amount of PCB 153 is less than 5.0 ng/g of the composition.

The composition is preferably for use in the treatment of patients diagnosed with NAFLD.

Further, the invention provides a method to increase the level of n-3 PUFAs or to correct a deficiency of n-3 PUFAs in a patient's blood, particularly in patients with NAFLD, without increasing the body burden of PCB 153.

### BRIEF DESCRIPTION OF THE DRAWINGS

FIG. 1a) and FIG. 1b) provide batch data graphs showing the amount of PCB 153 in two series of batches, respectively from 2010 and 2014-2015, for a high concentrate composition of EPA and DHA;

FIG. 2 is a bar graph showing an increase lipid accumulation in lipid treated HepG2 cells compared to control cells wherein HepG2 cells were exposed to Oleic acid: Palmitic acid (O:P), 1 mM in a 2:1 ratio, for 24 hours, or left untreated as control (C). Intracellular accumulation of lipids was detected and quantified by oil red O staining using spectrophotometry at wavelength 490 nm. OD: Optical density;

FIG. 3 is a bar graph showing a reduced accumulation of intracellular lipids in HepG2 cells after treatment with polyunsaturated fatty acids (PUFA), EPA, and DHA, wherein HepG2 cells were treated with EPA and DHA at a concentration of 100 μM (1:1) or with fatty acids-free bovine serum albumin (BSA) as a control, together with 1 mM Oleic/Palmitic acid, for 24 hours. Intracellular accumulation of lipids was detected and quantified by oil red O staining using spectrophotometry at wavelength 490 nm. OD: Optical density; and

FIG. 4 is a bar graph showing that a PCB 153 exposure diminishes the beneficial effect of PUFAs (EPA and DHA) on lipid accumulation in an experimental in vitro model of NAFLD, wherein steatic HepG2 cells (induced by the treatment of Oleic acid: Palmitic acid) and treated with PUFA (EPA and DHA), were exposed to PCB 153 dissolved in DMSO (stock solution 1 mM, final concentration 50 μM) or DMSO alone as control. Intracellular accumulation of lipids was detected and quantified by oil red O staining using spectrophotometry at wavelength 490 nM. OD: Optical density.

### DETAILED DESCRIPTION OF THE PREFERRED EMBODIMENTS

The present invention provides a new composition of n-3 PUFAs comprising particularly low amounts of the environmental pollutant PCB 153. Patients with a diagnosis of non-alcoholic fatty liver disease are likely to have very low levels of n-3 PUFAs in the blood, and often have an n-3 PUFA deficiency. By the term “deficiency”, it is meant that the level of n-3 PUFA is suboptimal for the patient group, either due to inadequate dietary consumption or due to an increased need for the nutrient due to a medical condition/disease. The composition and method of the invention have the ability to correct a nutritional deficiency in a target population. Hence, the compositions of the invention correct a nutritional deficiency of the marine n-3 PUFAs eicosapentaenoic acid (EPA) and docosahexaenoic acid (DHA) in patients diagnosed with non-alcoholic fatty liver disease. In NAFL, the disease is at the stage of simple steatosis, i.e. fat content of the liver is above 5% w/w, and without inflammation, therefore patients with NASH are not included in this definition. In order to correct the deficiency of n-3 PUFAs, rather high doses of a present composition should be administered to the patient over a long period, such as over several years and for instance for the rest of the life.

In one embodiment of the invention, the composition, method and use is directed to NAFL, i.e. the early phase of the NAFL disease (NAFLD), such as for treating steatosis. Preferably, the use in therapy or method of therapy according to the invention is directed to the steatosis phase, wherein the disease is reversible and has not progressed into NASH.

For high-fat treated mice there is a synergistic interaction between PCB 153 and a high-fat diet resulting in dramatically increased obesity and NAFLD, compared to a high fat diet alone. Hence, PCB 153 is a diet-dependent congener that worsens diet-induced NAFLD. This toxin changes the metabolic profile in high-fat treated mice and reduces the scavenging capacity in the liver, which is part of the pathophysiology in NAFLD. Therefore, it is important that any treatment for NAFLD patients should not expose the patients to a further contribution of PCB 153. Accordingly, an n-3 PUFA composition of the invention, which is preferably for use in the treatment of NAFLD, comprises an extremely low amount of PCB 153. Patients with NAFL generally have a low level of n-3 PUFAs in their blood, and when increasing this level and/or correcting the deficiency of the omega-3 by administering the composition of the invention over time, there will be no or a minimal contribution to the PCB 153 level in the blood from the composition. By using the composition of the invention, the ratio of n-3 PUFAs to PCB 153 may be increased. Accordingly, the composition of the invention is expected to have a particularly improved effect on NAFLD patients compared to similar compositions with higher amounts of PCB 153.

The n-3 PUFAs of the composition originate from an oil, particularly from a marine oil, and most particularly from fish oil, but may also be derived from algae oil, plant-based oil, or microbial oil. As used herein, the term “marine oils” includes oil from fish, shellfish (crustaceans), and sea mammals. Fish oil is the main source for human exposure to POPs, and the levels and specific congeners of PCBs found in human blood are linked to the amount and congener pattern in contaminated fish used for human consumption. Hence, because the human body burden of pollutants and specific congeners, including PCB 153, can be linked to the oil consumed, the quality of the omega-3 product matters.

A composition of the invention comprises a purified oil wherein the amount of the PCB 153 has been reduced to a minimal amount, and preferably wherein the oil has been further processed to the desired form and separated to feasible and useful concentrations. Non-limiting examples of fish oils that can be used as raw oil to manufacture the composition of the invention are, for example, Menhaden oil, Cod Liver oil, Herring oil, Capelin oil, Sardine oil, Anchovy oil, Salmon oil, Mackerel oil, Seal oil, and Krill oil. In one embodiment, the fish oil does not originate from Horse Mackerel. The fish oils mentioned above may be recovered from fish organs, e.g. cod liver oil, as well as from the meat of the fish or from the whole fish. The n-3 PUFAs of the final composition of the invention, including the EPA and DHA, may be in different forms, and are presented in at least one of free fatty acid form; esterified form, such as C1-C4 alkyl esters, and preferably ethyl ester; phospholipids; mono/di/tri-glycerides; and salts thereof. Preferably, the PUFAs are in esterified form, free acid form, or salt form, and more preferably in esterified form, and most preferably in ethyl ester form. All of these forms and derivatives are generally included when referring to n-3 PUFAs, or to EPA or DHA.

In one embodiment, the composition comprises at least 40% of at least one of EPA and DHA, and more preferably at least 45%, 55%, 65%, 75%, 85%, or 95% of at least one of EPA and DHA, by weight of the fatty acid in the composition. In one embodiment, the composition comprises high concentrations of one fatty acid, preferably either EPA or DHA. For instance, the composition may comprise at least 80% EPA, such as at least 90% EPA, e.g., about 97% EPA, by weight % of the fatty acids therein. In at least one embodiment, the composition comprises at least 60% of at least one of EPA and DHA by weight of the fatty acids therein, such as at least 75 weight %, such as at least 80%, such as about 84% of at least one of EPA and DHA by weight of the fatty acids in the composition. In one embodiment, the composition comprises no DHA. In another embodiment, the composition comprises no EPA.

In some embodiments of the present invention, the weight ratio EPA:DHA of the composition ranges from about 1:10 to about 10:1, from about 1:8 to about 8:1, from about 1:6 to about 6:1, from about 1:5 to about 5:1, from about 1:4 to about 4:1, from about 1:3 to about 3:1, or from about 1:2 to 2 about:1. In at least one embodiment, the weight ratio of EPA:DHA of the composition ranges from about 1:2 to about 2:1. In at least one embodiment, the weight ratio of EPA:DHA of the composition ranges from about 1:1 to about 2:1. In at least one embodiment, the weight ratio of EPA:DHA of the composition ranges from about 1.2:about 1.3.

In another embodiment, the composition is selected from specific mixtures of EPA and DHA, or derivatives thereof, such as selected from compositions comprising about 360 mg EPA and 240 mg DHA per g oil, 400 mg EPA and 200 mg DHA per g oil, 500 mg EPA and 200 mg DHA per g oil, 150 mg EPA and 500 mg DHA per g oil, 460 mg EPA and 380 mg DHA per g oil, above 900 mg EPA per g oil, above 900 mg DHA per g oil, and 97% EPA.

In a preferred embodiment, the composition for use according to the invention comprises an n-3 PUFA mixture of about 84 weight % EPA and DHA, preferably comprising 460 mg EPA-ethyl ester and 380 mg DHA-ethyl ester per gram, such as for the pharmaceutical named Omacor, Lovaza, or generics of these, wherein the amount of PCB 153 is less than 5.0 ng/g.

In addition to EPA and DHA, other polyunsaturated fatty acids, particularly omega-3 fatty acids, may be present in the composition. In one embodiment, the composition further comprises at least one fatty acid other than EPA and DHA. The at least one other fatty acid is, for example, selected from the group consisting of from a-linolenic acid (ALA), heneicosapentaenoic acid (HPA), docosapentaenoic acid (DPA), eicosatetraenoic acid (ETA), eicosatrienoic acid (ETE), stearidonic acid (SDA), hexadecatrienoic acid (HTA), tetracosapentaenoic, tetracosahexaenoic acid, and mixtures thereof. In one embodiment, the composition comprises DPA, such as up to 5 weight %, such as about 2 weight %. Some omega-6 fatty acids may be present, such as arachidonic acid or γ-linolenic acid, but the content is preferably kept low.

As there is a synergistic interaction between PCB 153 and high fat diet (HFD), obesity and NAFL are increased in patients exposed to PCB 153 compared to those without exposure, and PCB 153 and HFD, in combination, significantly change hepatic metabolism. The metabolite glutathione has been shown to be substantially decreased in HFD+PCB 153 compared to HFD alone. Oxidative stress and the function of hepatic mitochondria and reduced liver glutathione (GSH) play a role in the pathogenesis of NAFLD. However, omega-3 PUFAs reduce lipoperoxidation in steatic liver, reduce hepatic liver content, and increase GSH. This beneficial effect of n-3 PUFA however may be counteracted by the presence of PCB 153 unless using the composition of the invention. Hence, realizing that PCB 153 and omega-3 PUFAs affect the same metabolic processes, in opposite directions, it is important to provide an omega-3 composition for this patient group that does not include an amount of PCB 153 which outweighs the effect of omega-3 acids. Accordingly, the amount of PCB 153 in the composition of the invention is minimized. The amount of PCB 153 should be less than 5.0 ng/g composition. More preferably the amount of PCB 153 in the composition is less than 4.0 ng/g, less than 3.0 ng/g, less than 2.0 ng/g, or even less than 1.0 ng/g, such as less than 0.7 ng/g.

The compositions may further comprise at least one antioxidant. Examples of antioxidants suitable for the present composition include, but are not limited to, alpha-tocopherol (vitamin E), mixed tocopherol, calcium disodium EDTA, alpha tocoferyl acetates, butylhydroxytoluenes (BHT), and butylhydroxyanisoles (BHA). In a preferred embodiment, the composition includes mixed tocopherol. Mixed tocopherol typically includes all the forms alpha-, gamma-, and delta-tocopherol. In one embodiment, the composition does not include alpha-tocopherol as the sole antioxidant. The composition should include the antioxidant in at least an amount of 1 mg/g of the composition. In one embodiment, the composition includes the antioxidant in less than 3.0 mg/g of the composition. In one embodiment, the anti-oxidant, such as the mixed tocopherol, is from non-genetically modified organisms (GMO). Preferably, the composition comprises non-GMO mixed tocopherol.

In another aspect, the invention provides a method to increase the level of n-3 PUFAs or to correct a deficiency of n-3 PUFAs in NAFLD patients' blood. The method provides such increase or correction without contributing to the body burden of PCB 153, such as without contributing to an increase in the PCB 153 level in the patients' blood. The invention provides a method to increase the level of n-3 PUFAs or to correct a deficiency of n-3 PUFAs in the patients' blood, particularly in patients with NAFLD, wherein a composition comprising at least 40% of at least one of EPA and DHA, or derivatives thereof, by weigh of the fatty acid therein, and wherein the amount of PCB 153 in the composition is less than 5.0 ng/g, is administered to the patient.

The invention provides a method to increase the blood levels of marine n-3 PUFAs in patients diagnosed with NAFLD. The increase or correction of n-3 PUFA acheived by use of the method or composition of the invention can be quantified as a DHA and/or EPA enrichment in red blood cells (erythrocytes). In one embodiment, the change obtained in erythrocyte EPA and DHA, as a percentage of total fatty acids, by using the method of the invention is at least 10%, such as at least 20%, such as e.g., a 30-60% increase.

Alternatively, quantitative measurements can be made of the actual erythrocyte EPA and erythrocyte DHA. By the method of the invention, a substantial increase in the amount of erythrocyte EPA and erythrocyte DHA is acheived.

Alternatively, a quantification of DHA and EPA, or total PUFA, amount in plasma phospholipid or serum phospholipid can be measured. Also for these methods, blood tests are taken and the amount is measured, e.g., by gas chromatography. With reference to Lou et al, 2014, the invention provides a method wherein the total n-3 PUFA levels in serum phospholipid in blood is increased to above about 8.0 weight %, such as above about 10.0 weight % of the total fatty acids. The total n-3 PUFA level may be defined as the sum of the plasma or serum phospholipid eicosapentaenoic acid (EPA) and docosahexaenoic acid (DHA) levels in weight percentage (wt %) of total plasma or serum phospholipid fatty acids. The PUFA level, i.e., the sum of EPA and DHA, in addition to the level of the individual fatty acids, in blood samples may be measured by gas chromatography. Reference is also made to a cohort study reported by Eide et al, “The association between Marine n-3 polyunsaturated fatty acid level and survival after renal transplantation”, Clin J Am Soc Nephrol 10, June 2015, showing the PUFA level of a study population. The median level of marine n-3 PUFAs in plasma phospholipids was 7.95 wt %, similar to the levels found by Lou et al. The general conclusion of the study was that higher plasma phospholipid marine n-3 PUFA levels were associated with better patient survival. Based on these data, the applicant theorizes that the same levels of PUFAs in blood should be achieved for patients with NAFLD by use of a composition with a particularly low level of PCB 153. Accordingly, in one embodiment, the invention provides a method wherein the level of n-3 PUFAs in the plasma phospholipid in blood of a patient diagnosed with NAFLD is increased to above about 8.0 wt % by administration of a composition of the present invention. More preferably, the n-3 PUFA level is increased to above about 10.0 wt %.

Preferably, the increased level or the correction of nutritional deficiency will be evaluated as a significant increase in Omega-3 index, the percentage of marine omega-3 PUFAs to total fatty acids in red blood cells (erythrocytes). Red blood cell fatty acid composition, and hence the Omega-3 index, reflects a long term intake of marine omega-3 PUFAs, contrary to plasma measurements which are more influenced by the subject's diet over the last few days before sampling. At the same time, using the composition of the invention, the PCB 153 amount in the blood is minimally increased or is unchanged, although the level of omega-3 PUFAs is increased. Accordingly, by the method of the invention the ratio of omega-3 PUFAs to PCB 153 in the blood is expected to be increased. The Omega-3 Index expresses EPA+DHA as a percentage of the total fatty acid content of the red blood cells. Hence, in another embodiment the invention provides a method of treating a patient who is diagnosed with NAFLD, the method comprising administering to the patient a composition of the invention comprising n-3 PUFAs, in a sufficient amount and for a sufficient time, to correct a deficiency of said n-3 PUFAs or to increase the level in the blood, wherein the Omega-3 Index (%) is increased. Patients with a diagnosis of non-alcoholic fatty liver disease are likely to have very low levels of n-3 PUFAs in the blood, and they may have an Omega-3 index of about 3%, e.g. 3-4%, or even lower. By the treatment disclosed herein, using the method or the composition of the invention, the absolute value for the Omega-3 Index (%) for a patient is expected to be increased to above about 6.0%, more preferably above 7.0%, such as above 8.0%. In one embodiment, the Omega-3 index is increased with at least a fold increase of 10%, such as at least 20%, such as at least 40%, as a result of the treatment. Even a fold increase of 100% or more in the Omega-3 index is expected as a result of the treatment, particularly for those patients having very low levels before the treatment is started.

In another embodiment, the invention provides a method as disclosed to correct an imbalance in the ratio of n-6 PUFAs to n-3 PUFAs in the blood. In one embodiment, the invention provides a method wherein the ratio of n-6 PUFAs to n-3 PUFAs in the plasma phospholipid is corrected to a ratio of less than about 4.0 and more preferably less than 3.0.

Further, by using the composition or the method of the invention, patients diagnosed with NAFLD are likely to have a decrease in liver fat. An improvement in liver fat percentage is expected, and this is associated with a DHA and/or EPA enrichment. In one embodiment, the method of the invention provides at least 10% decrease in liver fat, more preferably at least 15% decrease in liver fat (hepatic steatosis), and more preferably at least 20% decrease in liver fat during the treatment. The results are dependent, for example, on the amount of liver fat when starting the treatment, the dosage of the composition, and how long the patient has been treated.

A clinical trial is being conducted to demonstrate the beneficial effects of the present invention. In this study, the effects of nutritional correction on risk factors associated with non-alcoholic fatty liver are assessed. End points for this study, in addition to those described above, are Liver enzymes ALT, AST, GGT; Total cholesterol; LDL-cholesterol; HDL-cholesterol; and Liver fat by MRI in a subset. In one embodiment of the invention, the effects achieved in this study are encompassed by the method of the invention.

In one or more embodiments of the invention, the method includes at least a step of measuring the level of n-PUFAs in the patient's blood. Such measuring may be done once or several times, and may be done regularly to check whether the level of PUFAs is at an acceptable level, or to decide on a further treatment, such as the dose to be administered. The PUFA level may be measured in the plasma phospholipids, in the serum phospholipid, e.g. by gas chromatography, or alternatively the level of PUFAs may be measured in the red blood cells. The method may further include steps to calculate the percentage increase of the individual or total n-3 PUFAs. In one embodiment, the dose of the composition is adjusted based on the result of the measurement in order to correct the PUFA deficiency, increase the PUFA level, or correct an imbalance of omega-6:omega-3 ratio.

The total daily dosage of the composition may range from about 0.600 g to about 6.000 g. For example, in some embodiments, the total dosage of the composition ranges from about 0.800 g to about 4.000 g, from about 1.000 g to about 4.000 g, such as 3.000 g, or from about 1.000 g to about 2.000 g.

The composition may be administered in from 1 to 10 dosages, such as from 1 to 4 times a day, such as once, twice, three times, or four times per day, and further for example, once, twice or three times per day. The administration may be oral or any other form of administration that provides a dosage of n-3 PUFAs to a subject. In a preferred embodiment, the subject is administered with capsules of 1 g three times a day, preferably wherein the capsules each comprise 460 mg EPA-ethyl ester and 380 mg DHA-ethyl ester. In one embodiment, the dose is adjusted according to the level of n-3 PUFAs measured for the individual patient. The composition is preferably administered over a long period, such as 12-52 weeks, e.g. 24-46 weeks. An adequate level of n-3 PUFAs is expected to be reached after 12-16 weeks, but the patient should continue the treatment to maintain this level. In one embodiment, the patient should continue to take the composition for the rest of the life. By administering the disclosed compositions, with the low content of PCB 153, in doses and a time period as disclosed, the therapeutic results described above are forseen.

The composition has the ability to reduce the risk factors associated with NAFLD. Particularly, by using the composition or the method of the invention, the liver function can be improved by reducing the hepatic steatosis and/or normalize hepatic lipid metabolism and/or correct adipocytokines, reduce risk of insulin resistance, metabolic syndrome, chronic liver disease, hepatocellular carcinoma, type 2 diabetes, and cardiovascular diseases. In one embodiment, the method of the invention reverses NAFL and hence may reduce the risk that the disease develops into NASH, cirrhosis, or liver failure.

In another aspect, the invention provides a composition comprising at least 40 weight % of at least one of EPA and DHA, or derivatives thereof, and wherein the amount of PCB 153 is less than 5.0 ng/g, for use in therapy of patients diagnosed with NAFLD. This aspect includes the same embodiments as outlined above for the first two aspects. Hence, the use provides an increased level of n-3 PUFAs or corrects a deficiency of n-3 PUFAs in the blood of the patient, as earlier described.

In some embodiments of the present disclosure, the composition acts as an active pharmaceutical ingredient (API). In some embodiments, the fatty acid of the composition is present in a pharmaceutically-acceptable amount. As used herein, the term “pharmaceutically-effective amount” means an amount sufficient to treat, e.g., reduce and/or alleviate the effects, symptoms, etc., at least one health problem in a subject in need thereof. In at least some embodiments of the present invention, the composition does not comprise an additional active agent. In this embodiment, the composition may be used in a pharmaceutical treatment of patients diagnosed with NAFLD. When the composition is a pharmaceutical composition, the composition preferably comprises at least 75% of at least one of EPA and DHA by weight of the composition. For example, in one embodiment, the composition comprises at least 80% EPA and DHA by weight of the composition, such as at least 85%, at least 90%, or at least 95%, by weight of the composition.

In another embodiment, the composition according to the invention is a food supplement or a nutritional supplement comprising at least one of EPA and DHA. In a related embodiment, the invention provides a composition selected from the group of Enteral Formulas for Special Medical Use, Foods for Specified Health Uses, Food for Special Medical Purposes (FSMP), Food for Special Dietary Use (FSDU), Medical Nutrition, and a Medical Food. Such a composition is particularly suited for patients having a deficiency of certain nutrients, such as n-3 PUFAs. The composition is suited for a nutritional management of NAFLD patients having a distinctive nutritional requirement. Such a composition typically is administered to the subject under medical supervision. In this embodiment, the composition comprises the relevant n-3 PUFAs, particularly EPA and/or DHA as disclosed in the first aspect, to increase or correct the level of the n-3 PUFAs in the blood of a patient diagnosed with NAFLD. Accordingly, the composition for use in the treatment of a patient who has NAFLD is selected from the above group. In a preferred embodiment, the composition is, or forms part of, Medical Food suitable for administration to NAFLD patients. The composition and the method of the invention have the ability to correct a nutritional deficiency in a target population. The invention hence provides nutritional correction of risk factors associated with NAFLD. Also, when using a present composition in the nutritional management of NAFLD, e.g., as a Medical Food, it is preferred that the composition is a highly concentrated composition of EPA and/or DHA. Preferably, the composition comprises at least 55% of at least one of EPA and DHA by weight of the composition.

The compositions presently disclosed may be administered, for example, in capsule, tablet, or any other drug delivery form. For example, the composition may be encapsulated, such as in a gelatin capsule. Formulated forms of each at the above are all encompassed by the definition of the composition. Examples of such formulations are Self Micro Emulsifying Drug Delivery Systems (SMEDDS), Self Nano Emulsifying Drug Delivery Systems (SNEDDS), and Self-Emulsifying Drug Delivery Systems (SEDDS) which form an emulsion in an aqueous solution. For example, the composition may be in the form of a pre-concentrate of any of the above which spontaneously form an emulsion when mixed with gastric/intestinal fluid. Such emulsions, when formed, may provide for increased or improved stability of the fatty acids for uptake in the body and/or provide increased surface area for absorption. Further, the composition may be in the form of emulsions and formulations where the active/nutritional ingredient is microencapsulated or in the form of a gel or semi-solid formulations

A composition of the invention is prepared by a process wherein the PCB 153 is removed from crude fish oil. The process may include a stripping process as outlined in the WO2004/007654, designating the United States and incorporated herein by reference. As outlined in WO2004/007654, such stripping process includes a thin-film evaporation process, a molecular distillation, or a short-path distillation of a fatty acid oil mixture using a volatile working fluid. The volatile working fluid either may be mixed with the fatty acid oil mixture to be purified or may be added in the stripping process separately. The volatile working fluid comprises at least one of a fatty acid ester, a fatty acid amide, and a free fatty acid. PCB 153 is stripped off together with the volatile working fluid. Such process further includes optimized steps to remove the PCB153, preferably including an adjusted feed rate through the stripping column. Particularly when preparing a composition comprising high concentrations of EPA and/or DHA, such as at least 60% of at least one of EPA and DHA by weight of the fatty acids therein, the low level of PCB 153 is achievable by e.g., reducing the feed rate through the stripping column. In one embodiment, the feed rate of the fatty acid oil mixture to be stripped is 10-80 kg/h·m². A reduced feed rate has been found to remove more of the PCB 153 pollutant. Even though the production capacity may be reduced as a consequence, the benefit of obtaining a very pure oil, is highly favorable. Alternatively, the stripping process conditions may be modified by increasing the temperature, preferably within the range of 200-240° C., or by stripping more than once. The conditions and parameters of the stripping process is dependent on several parameters including the composition of the feed, the apparatus available, and particularly which product composition is to be prepared. In one embodiment, the stripping process to remove pollutants, preparing a purified oil intermediate with a reduced PCB153 content, is followed by a trans-esterification process. Preferably, the stripping processing step is followed by the steps of subjecting the stripped marine oil mixture to at least one trans-esterification reaction with a C1-C6 alcohol under substantially anhydrous conditions, and in the presence of a suitable catalyst (a chemical catalyst or an enzyme) to convert the fatty acids present as triglycerides in the marine oil mixture into esters of the corresponding alcohol. Thereafter, the product obtained may be purified, i.e., by separation of the fatty acids and recovery of the wanted n-3 PUFAs. This may for example include distillations, preferably one or more molecular distillations, or alternatively by other methods, such as chromatographic separations. The final composition, preferably after esterification and recovery, comprises the claimed low amount of PCB 153.

Preferred embodiments of the invention are now described by way of example only and with reference to the accompanying drawings, in which Figures la and b provide batch data from analysis of the amount of PCB 153 in two series of batches, respectively from 2010 and 2014-2015, for a high concentrate composition of EPA and DHA.

### EXAMPLES

### Example 1

### Measurement of PCB 153 in Compositions of EPA and DHA

The amount of PCB 153 in highly concentrated compositions of EPA and DHA ethyl esters was quantified, using the method described below, in batches prepared in 2010 and 2014-2015, to evaluate the development in the manufacturing process and to assess the effectiveness of the purification step (stripping). About 5 g of the compositions were dissolved in n-hexane and ¹³C-labeled quantification standards for PCBs were added. After a clean-up with acidic silica gel and alumina oxide, the ¹³C-labelled injection standards were added. The measurements of PCB 153 amount were performed on a High Resolution Gas Chromatography/High Resolution Mass Spectrometry system. Calculations were done with the isotope dilution method using one ¹³C-labelled standard for each native congener of interest, including for PCB 153.

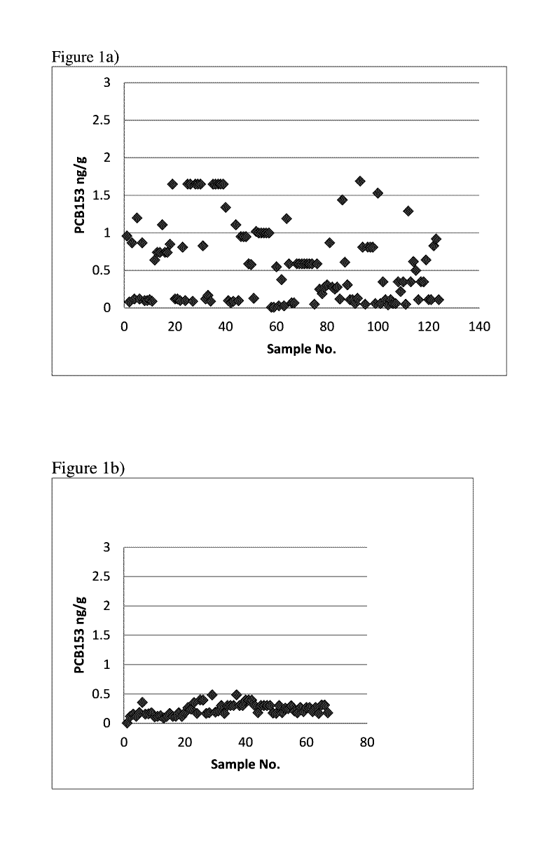

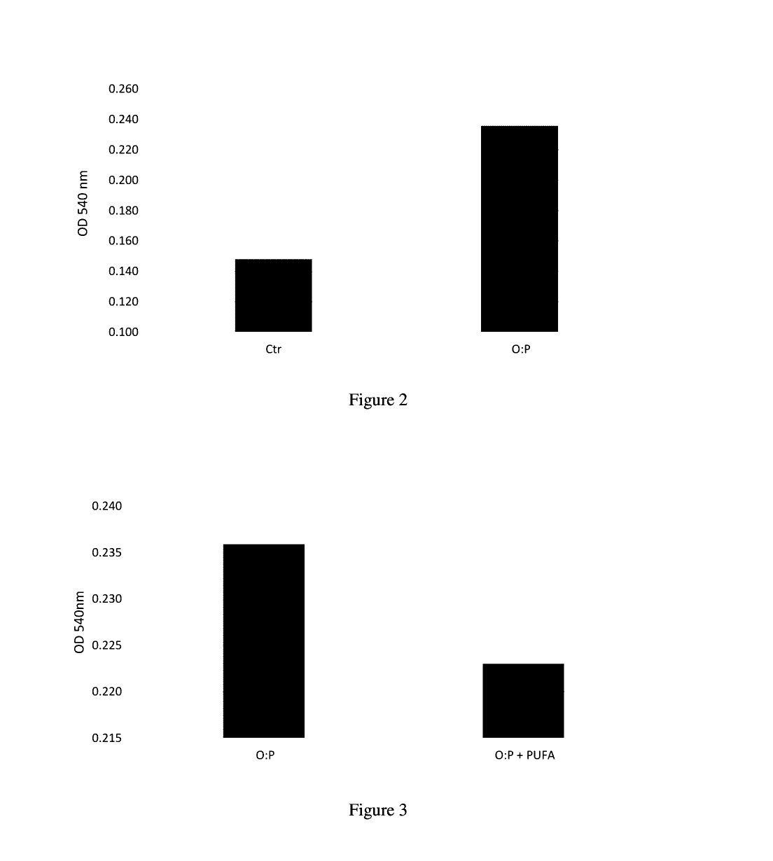

The results, FIGS. 1a and b, show that the optimization process results in lower levels of PCB 153 in batches manufactured in 2014-2015 compared to batches produced in 2010. In the compositions from 2014-2015, the amount of PCB 153 was consistently below 1.0 ng/g.

### Example 2

### In Vitro Assessment of Omega-3 Effect on Seatic HepG2 Cells with and without PCB 153 Exposure

An experimental model of hepatocellular steatosis with a fat over-accumulation profile in vitro model of human NAFLD in HepG2 cells was established by lipid exposure to cells in vitro thereby inducing significant intracellular fat accumulation in the absence of overt cytotoxicity. Palmitic (C16:0) and oleic (C18:1) acids are the most abundant fatty acids in liver triglycerides in both normal subjects and patients with NAFLD. The human hepatocyte-derived cell line HepG2 cells were seeded in a 96 well plate at a density of 5000 cells/well on Day 1. The cells were treated with the combination of Oleic acid-Palmitic acid (1 mM in a 2:1 ratio), at day 2 for 24 hours as earlier described by Gòmez-Lechòn et al. “A human hepatocellular in vitro model to investigate steatosis”, Chemico-Biological Interactions 165 (2007): 106-116. Intracellular accumulation of lipids was detected and quantified by oil red O staining using spectrophotometry at wavelength 490 nm. Oil red O color intracellular lipids droplets after cell fixation with paraformaldehyde. Absence of cell death was confirmed using a colometric lactate dehydrogenase (LDH) assay. Lactate dehydrogenase (LDH) is a cytosolic enzyme present in many different types of cells. When the plasma membrane is damaged, LDH is released into cell culture media. The released LDH was quantified by a coupled enzymatic reaction using a LDH cytotoxicity kit, and following the procedure according to the manufacture's instruction. The results show that lipid exposure of HepG2 cells lead to increased intracellular fat accumulation compared to untreated control cells in the absence of overt cytotoxicity representing an experimental model for NAFLD (FIG. 2).

To assess the effect of n-3 PUFA on experimental NAFLD, the HepG2cells were treated with EPA and DHA at a concentration of 100 μM (1:1), or with fatty acids-free bovine serum albumin (BSA), as a control, together with 1 mM Oleic/Palmitic acid for 24 hours. BSA was used as a control because the fatty acid stock solutions were prepared on fatty acid-free BSA in the ratio 6 mM fatty acid/2.4 mM BSA. Intracellular accumulation of lipids was detected and quantified by oil red O staining using spectrophotometry at wavelength 490 nm. Accumulation of intracellular lipids was reduced upon treatment with EPA and DHA (FIG. 3).

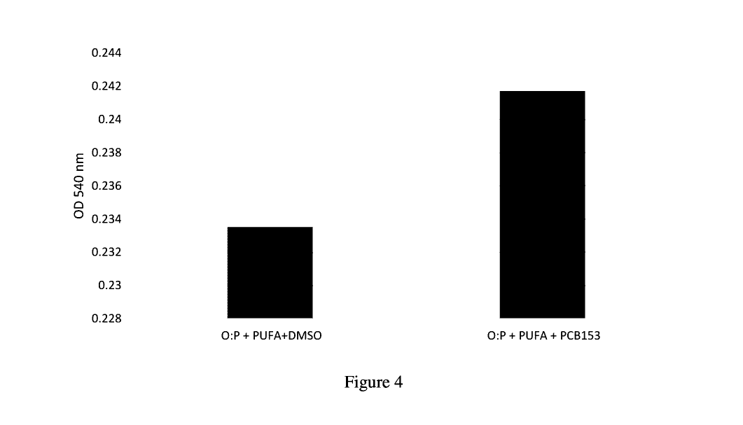

Steatic HepG2 cells, with and without EPA/DHA treatment, as described above, were exposed to PCB 153 dissolved in DMSO (stock solution 1 mM, final concentration 50 μM) or DMSO alone as control, EPA/DHA and PCB 153 were added together with Oleic/Palmitic acid, 24 hours after plating, and the cells were treated for 24 hours at 37° C. An accumulation of intracellular lipids was quantified using lipid oil red O staining as described above. PCB 153 exposure was found to diminish the beneficial effect of n-3 PUFA (EPA/DHA) on the development of steatosis in an experimental in vitro model of NAFLD (FIG. 4).

1. A method of treating a patient diagnosed with non-alcoholic fatty liver disease (NAFLD) comprising administering an n-3 polyunsaturated fatty acids (n-3 PUFAs) composition to the patient in a sufficient amount to increase a level of n-3 PUFAs or to correct a deficiency of n-3 PUFAs in the blood of the patient, wherein the composition comprises at least 40 weight % of at least one of eicosapentaenoic acid (EPA) and docosahexaenoic acid (DHA), or derivatives thereof, by weight of the fatty acids therein, and an amount of PCB 153 in the composition is less than 5.0 ng/g.

2. The method of claim 1 wherein the composition comprises at least 60% of at least one of EPA and DHA by weight of the fatty acids therein.

3. The method of claim 1 wherein a weight ratio of EPA:DHA in the composition ranges from about 1:10 to about 10:1.

4. The method of claim 1 wherein the composition originates from a marine oil.

5. The method of claim 1 wherein the PUFAs are present in at least one of a free fatty acid form; an esterified form; a phospholipid form; a mono/di/tri-glyceride form, and salts thereof.

6. The method of claim 1 wherein the composition comprises a sum of about 84 weight % EPA and DHA.

7. The method of claim 1 wherein the composition comprises 460 mg EPA-ethyl ester and 380 mg DHA-ethyl ester per gram.

8. The method of claim 1 wherein the amount of PCB 153 is less than 4.0 ng/g.

9. The method of claim 8 wherein the amount of PCB 153 is less than 3.0 ng/g.

10. The method of claim 9 wherein the amount of PCB 153 is less than 1.0 ng/g.

11. The method of claim 10 wherein the amount of PCB 153 is less than 0.7 ng/g.

12. The method of claim 1 wherein the increase of n-3 PUFAs in the blood is at least 10%, measured as a change obtained in erythrocyte EPA and DHA as a percentage of total fatty acids.

13. The method of claim 1 wherein a level of n-3 PUFAs in blood is increased to above about 8.0 wt % of total plasma or serum phospholipid fatty acid level in the blood.

14. The method of claim 1 wherein an Omega-3 Index (%) is increased by at least 20%.

15. The method of claim 1 wherein the composition is selected from the group of Enteral Formulas for Special Medical use, Foods for Specified Health Uses, Food for Special Medical Purposes (FSMP), Food for Special Dietary Use (FSDU), Medical Nutrition, and a Medical food.

16. The method of claim 1 wherein an Omega-3 Index (%) is increased to above about 6.0% of the total fatty acids in the blood.

17. The method of claim 1 wherein the method corrects an imbalance in a ratio of n-6 PUFAs to n-3 PUFAs in the blood.

18. The method of claim 1 wherein the composition is administered to the patient over a sufficient time to increase a level of 3-PUFAs or correct a deficiency of 3-PUFAs in the blood of the patient.

19. An n-3 polyunsaturated fatty acids (PUFAs) composition comprising at least 40% of at least one of EPA and DHA, or derivatives thereof, by weight of the fatty acids therein, wherein an amount of PCB 153 in the composition is less than 5.0 ng/g.

20. The composition of claim 19 further comprising an antioxidant.

21. The composition of claim 20 wherein the antioxidant comprises mixed tocopherol.

22. The composition of claim 19 comprising at least 60% of at least one of EPA and DHA, by weight of the fatty acids therein.

23. The composition of claim 19 wherein a weight ratio of EPA:DHA in the composition ranges from about 1:10 about 10:1.

24. The composition of claim 19 wherein the PUFAs are present in at least one of a free fatty acid form; an esterified form; a phospholipid form; a mono/di/tri-glyceride form, and salts thereof.

25. The composition of claim 19 wherein the amount of PCB 153 in the composition is less than 2.0 ng/g.

26. The composition of claim 25 wherein the amount of PCB 153 in the composition is less than 0.7 ng/g.
